(19) United States
(12) Patent Application Publication
(10) Pub. No.: US 2025/0352206 A1
(43) Pub. Date: Nov. 20, 2025

(54) ARTICULATION MECHANISM FOR SURGICAL STAPLING DEVICE

(71) Applicant: Covidien LP, Mansfield, MA (US)

(72) Inventor: Justin P. WILLIAMS, North Haven, CT (US)

(21) Appl. No.: 18/871,956

(22) PCT Filed: Jun. 1, 2023
(86) PCT No.: PCT/IB2023/055643

§ 371 Date: Dec. 5, 2024

Related U.S. Application Data
(60) Provisional application No. 63/349,619, filed on Jun. 7, 2022.

(51) Int. Cl. A61B 17/072; A61B 17/00

(52) U.S. Cl. CPC A61B 17/07207; A61B 2017/00477

(57) ABSTRACT

A surgical device includes an elongate body defining a longitudinal axis and a tool assembly that is pivotally attached to the elongate body for articulation about an articulation axis that is transverse to the longitudinal axis. The surgical device includes an articulation mechanism and a drive assembly. The drive assembly is movable about the articulation axis to actuate the tool assembly. The articulation mechanism includes proximal and distal drive links and proximal and distal driven links. The links are configured to guide and support the drive assembly to facilitate greater degrees of articulation.

### FIELD

This disclosure is generally related to surgical devices for endoscopic use and, more specifically, to a surgical device including an articulation mechanism for articulating a tool assembly of the surgical device.

### BACKGROUND

Various types of surgical devices used to endoscopically treat tissue are known in the art, and are commonly used, for example, for closure of tissue or organs in transection, resection, and anastomoses procedures, for occlusion of organs in thoracic and abdominal procedures, and for electrosurgically fusing or sealing tissue.

One example of such a surgical device is a surgical stapling device. Surgical stapling devices include a tool assembly having an anvil assembly and a cartridge assembly, and a drive assembly that is movable through the tool assembly. Typically, the drive assembly includes a flexible drive beam and a clamp member that is supported on a distal end of the flexible drive beam. The drive assembly is movable to advance the clamp member through the tool assembly to approximate the cartridge and anvil assemblies and to advance an actuation sled through the cartridge assembly to eject staples from the cartridge assembly.

During laparoscopic or endoscopic surgical procedures, access to a surgical site is achieved through a small incision or through a narrow cannula inserted through a small entrance wound in a patient. Because of limited area available to access a surgical site, many endoscopic devices include mechanisms for articulating the tool assembly of the device about a pivot axis to better access tissue. Typically, mechanisms that allow for greater degrees of articulation increase dead space of the tool assembly, i.e., the space between the pivot axis and the beginning of a staple line of the tool assembly. Increased dead space increases the length of the tool assembly and is undesirable.

A continuing need exists in the art for an articulating mechanism for a surgical device that minimizes dead space in the tool assembly but allows for greater degrees of articulation of the tool assembly.

### SUMMARY

This disclosure is directed to a surgical device that includes an elongate body defining a longitudinal axis and tool assembly that is pivotally attached to the elongate body for articulation about an articulation axis that is transverse to the longitudinal axis. The surgical device includes an articulation mechanism and a drive assembly. The drive assembly is movable about the articulation axis to actuate the tool assembly. The articulation mechanism includes proximal and distal drive links and proximal and distal driven links. The links are configured to guide and support the drive assembly to facilitate greater degrees of articulation.

Aspects of this disclosure are directed to a surgical device having an elongate body, a tool assembly, a drive assembly, and an articulation mechanism. The elongate body defines a first longitudinal axis and has a proximal portion and a distal portion. The tool assembly defines a second longitudinal axis and is supported on the distal portion of the elongate body for pivotal movement about an articulation axis between a non-articulated position and articulated positions. The articulation axis is transverse to the first and second longitudinal axes. The drive assembly includes a flexible drive beam and a clamp member. The flexible drive beam has a proximal portion and a distal portion. The clamp member is supported on the distal portion of the flexible drive beam and is received within the tool assembly. The drive assembly is movable between a retracted position and an advanced position to move the clamp member through the tool assembly. The articulation mechanism includes a proximal drive link, a distal drive link, a proximal driven link, and a distal driven link. The proximal drive link has a planar inner surface, a proximal portion, and a distal portion. The distal drive link has a proximal portion pivotally coupled to the distal portion of the proximal drive link and a distal portion pivotally coupled to the tool assembly. The proximal driven link has a planar inner surface, a proximal portion, and a distal portion. The distal driven link has a proximal portion pivotally coupled to the proximal portion of the proximal driven link and a distal portion pivotally coupled to the tool assembly. The proximal drive link is movable from an intermediate position to an advanced position to articulate the tool assembly about the articulation axis in a first direction and movable from the intermediate position to a retracted position to articulate the tool assembly about the articulation axis in a second direction. The planar inner surfaces of the proximal drive link and the proximal driven link define a channel through which the flexible drive beam moves when the drive assembly is moved between the retracted and advanced positions.

In aspects of the disclosure, the proximal drive link and the proximal driven link are confined to linear movement within the elongate body.

In some aspects of the disclosure, the planar inner surface of the proximal drive link is positioned to engage the flexible drive beam adjacent the articulation axis when the tool assembly is articulated in the first direction.

In certain aspects of the disclosure, the planar inner surface of the proximal driven link is positioned to engage the flexible drive beam adjacent the articulation axis when the tool assembly is articulated in the second direction.

In aspects of the disclosure, the distal drive link and the distal driven link have inner guide surfaces, and the inner guide surface of the distal drive link is positioned to engage the flexible drive beam adjacent the articulation axis when the tool assembly is articulated in the first direction.

In some aspects of the disclosure, the inner guide surface of the distal driven link is positioned to engage the flexible drive beam adjacent the articulation axis when the tool assembly is articulated in the second direction.

In certain aspects of the disclosure, the distal drive link and the proximal drive link are formed from a rigid material.

In aspects of the disclosure, the surgical device includes flexible stabilizing members positioned on each side of the flexible drive beam.

In some aspects of the disclosure, each of the flexible stabilizing members has a distal end coupled to the tool assembly and a proximal end received within the elongate body.

In certain aspects of the disclosure, the surgical device includes a handle assembly that is coupled to the proximal portion of the elongate body.

In aspects of the disclosure, the surgical device includes a mounting assembly that is fixedly coupled to the tool assembly and pivotally coupled to the elongate body.

In some aspects of the disclosure, the distal portions of the distal drive link and the distal driven link are pivotally coupled to the mounting assembly.

In certain aspects of the disclosure, the mounting assembly defines a channel, and the flexible drive beam extends through the channel of the mounting assembly.

In aspects of the disclosure, the proximal drive link and the proximal driven link define slots, and the distal drive link and the distal driven link are at least partly received within the slots.

Other aspects of the disclosure are directed to a reload assembly that includes a proximal body portion, a tool assembly, a drive assembly, and an articulation mechanism. The proximal body portion defines a first longitudinal axis and has a proximal portion and a distal portion. The proximal body portion is configured to releasably engage a surgical device. The tool assembly defines a second longitudinal axis and is supported on the distal portion of the proximal body portion for pivotal movement about an articulation axis between a non-articulated position and articulated positions. The articulation axis is transverse to the first and second longitudinal axes. The drive assembly includes a flexible drive beam and an I-beam. The flexible drive beam has a proximal portion and a distal portion. The I-beam is supported on the distal portion of the flexible drive beam and is received within the tool assembly. The drive assembly is movable between a retracted position and an advanced position to move the I-beam through the tool assembly. The articulation mechanism includes a proximal drive link, a distal drive link, a proximal driven link, and a distal driven link. The proximal drive link has a planar inner surface, a proximal portion, and a distal portion. The distal drive link has a proximal portion pivotally coupled to the distal portion of the proximal drive link and a distal portion pivotally coupled to the tool assembly. The proximal driven link has a planar inner surface, a proximal portion, and a distal portion. The distal driven link has a proximal portion pivotally coupled to the proximal portion of the proximal driven link and a distal portion pivotally coupled to the tool assembly. The proximal drive link is movable from an intermediate position to an advanced position to articulate the tool assembly about the articulation axis in a first direction and movable from the intermediate position to a retracted position to articulate the tool assembly about the articulation axis in a second direction. The planar inner surfaces of the proximal drive link and the proximal driven link define a channel through which the flexible drive beam moves when the drive assembly is moved between the retracted and advanced positions.

Other aspects of the disclosure are directed to a surgical device including an elongate body, a tool assembly, a drive assembly, and an articulation mechanism. The elongate body defines a first longitudinal axis and has a proximal portion and a distal portion. The tool assembly defines a second longitudinal axis and is supported on the distal portion of the elongate body for pivotal movement about an articulation axis between a non-articulated position and articulated positions. The articulation axis is transverse to the first and second longitudinal axes. The drive assembly includes a flexible drive beam and a clamp member. The flexible drive beam has a proximal portion and a distal portion. The clamp member is supported on the distal portion of the flexible drive beam and is received within the tool assembly. The drive assembly is movable between a retracted position and an advanced position to move the clamp member through the tool assembly. The articulation mechanism includes a drive link and a driven link. The drive link has a distal portion, a proximal portion, and a planar inner surface extending between the proximal and distal portions. The distal portion is pivotally coupled to the tool assembly. The driven link has a proximal portion, a distal portion, and a planar inner surface extending between the proximal and distal portion of the driven link. The planar inner surfaces of the drive link and the driven link define a linear channel through which the flexible drive beam moves when the drive assembly is moved between the retracted and advanced positions. The drive link is movable from an intermediate position to an advanced position to articulate the tool assembly about the articulation axis in a first direction and movable from the intermediate position to a retracted position to articulate the tool assembly about the articulation axis in a second direction.

Other features of the disclosure will be appreciated from the following description.

### BRIEF DESCRIPTION OF THE DRAWINGS

Various aspects of the disclosure are described herein below with reference to the drawings, wherein:

FIG. 1 is a side perspective view of a surgical stapling device according to aspects of the disclosure with a tool assembly of the stapling device illustrated in a non-articulated, open position;

FIG. 2 is a side perspective view of a distal portion of the surgical stapling device shown in FIG. 1 with the tool assembly shown articulated seventy degrees in a first direction;

FIG. 3 is a side perspective view of a distal portion of the surgical stapling device shown in FIG. 1 with the tool assembly shown articulated seventy degrees in a second direction;

FIG. 4 is a side perspective, exploded view of a reload assembly of the surgical stapling device shown in FIG. 1;

FIG. 5 is a side perspective view of a distal portion of the reload assembly shown in FIG. 4 partially assembled in the non-articulated position with an outer tube, upper housing portion, blowout plates, and drive beam removed from the reload assembly;

FIG. 6 is a top view of the reload assembly shown in FIG. 5 in the non-articulated position with the anvil, the outer tube, and the upper housing portion removed;

FIG. 7 is an enlarged view of the indicated area of detail shown in FIG. 6;

FIG. 8 is top, cutaway view of a central portion of the reload assembly shown in FIG. 6 with the anvil, the outer tube, and the upper housing portion removed and the tool assembly articulated seventy degrees in the second direction; and

FIG. 9 is top, cutaway view of a central portion of the reload assembly shown in FIG. 6 with the anvil, the outer tube, and the upper housing portion removed and the tool assembly articulated seventy degrees in the first direction.

### DETAILED DESCRIPTION

The disclosed surgical stapling device will now be described in detail with reference to the drawings in which like reference numerals designate identical or corresponding elements in each of the several views. However, it is to be understood that aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary manner, while the term “distal” is used generally to refer to that portion of the device that is farther from the clinician during use of the device in its customary manner. In addition, the term “endoscopic” is used generally to refer to endoscopic, laparoscopic, arthroscopic, and/or any other procedure conducted through a small diameter incision or cannula, and the term “clinician” is used generally to refer to medical personnel including doctors, nurses, and support personnel. Further, directional terms such as “front”, “rear”, “upper”, “lower”, “top”, “bottom”, and similar terms are used to assist in understanding the description and are not intended to limit the disclosure.

The disclosed surgical device includes an elongate body defining a longitudinal axis and a tool assembly that is pivotally attached to the elongate body for articulation about an articulation axis that is transverse to the longitudinal axis. The surgical device includes an articulation mechanism and a drive assembly that is movable about the articulation axis to actuate the tool assembly. The articulation mechanism includes proximal and distal drive links and proximal and distal driven links. The links are configured to guide and support the drive assembly to facilitate greater degrees of articulation.

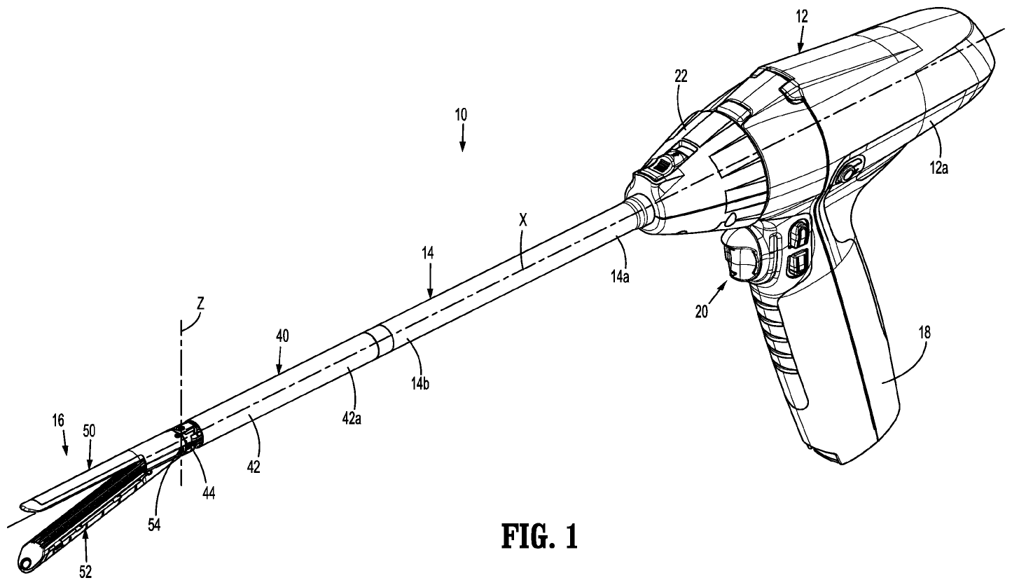

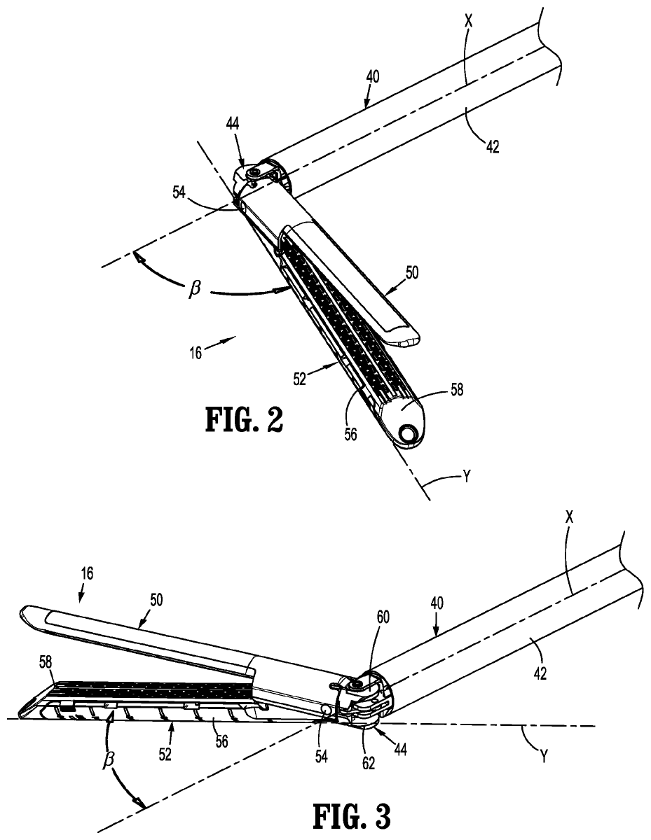

FIGS. 1-3 illustrate a surgical device according to aspects of the disclosure shown generally as surgical device 10. The surgical device 10 includes a handle assembly 12, an elongate body 14, and a tool assembly 16. The elongate body 14 defines a longitudinal axis “X” (FIG. 1) and the tool assembly 16 defines a longitudinal axis “Y” (FIG. 2). The tool assembly 16 is pivotally coupled to the elongate body 14 and can pivot between a non-articulated position (FIG. 1) in which the longitudinal axes “X” and “Y” of the elongate body 14 and tool assembly 16 are coaxial (FIG. 1) to articulated positions in which the longitudinal axes “X” and “Y” of the elongate body 14 and tool assembly 16 are misaligned with each other (FIGS. 2 and 3) to define acute angles “B”.

The handle assembly 12 includes a body 12a that forms a stationary handle 18 and actuation buttons 20 that are operable to initiate operation of the surgical device 10, i.e., approximation of the tool assembly 16, articulation of the tool assembly 16, and firing of staples from the tool assembly 16. In aspects of the disclosure, the handle assembly 12 supports a rotation knob 22 that is coupled to a proximal portion 14a of the elongate body 14 and is rotatable to rotate the elongate body 14 and the tool assembly 16 in relation to the handle assembly 12 about the longitudinal axis “X”. While the surgical device 10 may be configured to fire staples, it is contemplated that the surgical device 10 may be adapted to fire any other suitable fasteners such as clips and two-part fasteners. Although the surgical device 10 is illustrated as a surgical stapling device 10, it is also envisioned that certain components described herein may be adapted for use in other types of articulating endoscopic surgical instruments including endoscopic forceps, graspers, dissectors, other types of surgical stapling instruments, powered vessel sealing devices and/or cutting devices.

In aspects of the disclosure, the tool assembly 16 forms part of a reload assembly 40 that is releasably coupled to the elongate body 14 and can be replaced to facilitate reuse of the stapling device 10. The reload assembly 40 includes a proximal body portion 42, the tool assembly 16, and a mounting assembly 44 that pivotably couples the tool assembly 16 to the distal portion of the proximal body portion 42. The proximal body portion 42 is coaxial with the longitudinal axis “X” of the elongate body 14 and has a proximal portion 42a that is releasably coupled to a distal portion 14b of the elongate body 14. It is envisioned that the tool assembly 16 can be pivotably secured to the elongate body 14 via the mounting assembly 44 and need not form part of a reload assembly 40, i.e., the elongate body 14. It is also envisioned that the mounting assembly 44 can be integrally formed with the tool assembly 16.

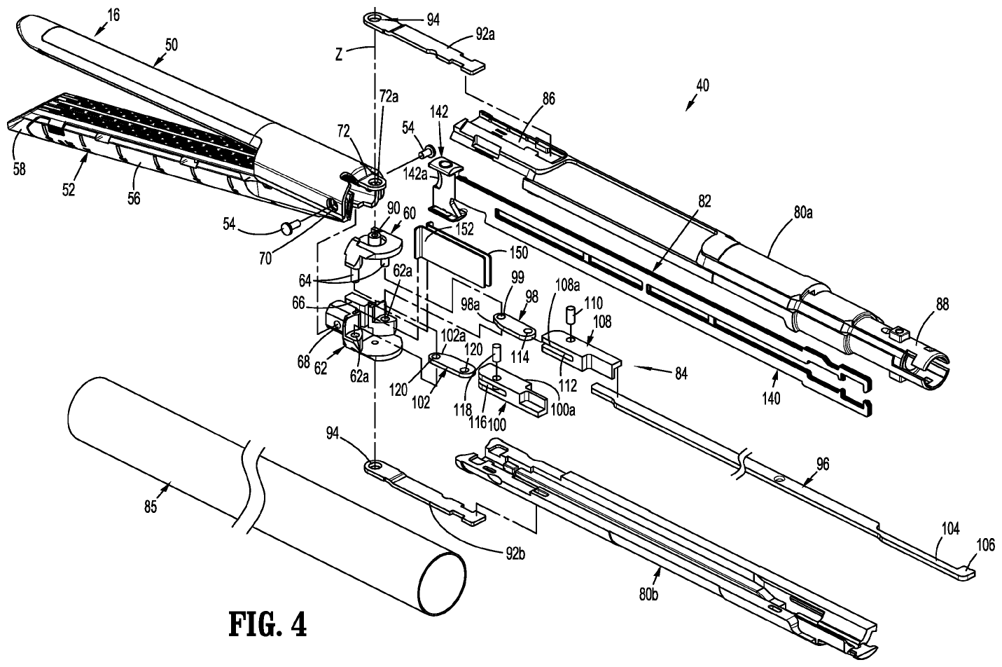

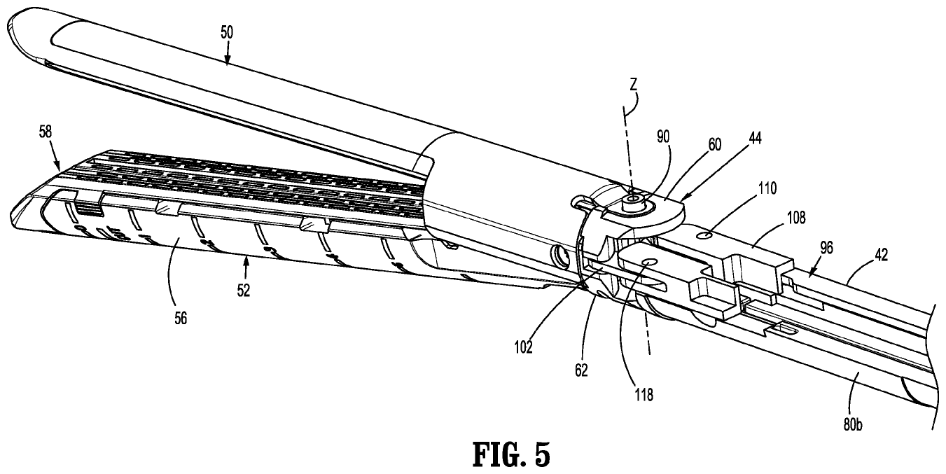

FIGS. 4 and 5 illustrate the reload assembly 40 which includes the tool assembly 16, the proximal body portion 42, and the mounting assembly 44. The tool assembly 16 includes an anvil assembly 50 and a cartridge assembly 52. The anvil assembly 50 is coupled to the cartridge assembly 52 by pivot members 54 (FIG. 3) that facilitate movement of the cartridge assembly 52 in relation to the anvil assembly 50 between the open position (FIG. 1) and the clamped position. The cartridge assembly 52 includes a channel member 56 and a staple cartridge 58. The channel member 56 defines a cavity that receives the staple cartridge 58. In aspects of the disclosure, the staple cartridge 58 is releasably received within the channel member 56 and can be replaced to facilitate reuse of the stapling device 10 (FIGS. 1 and 2). Alternately, it is envisioned that the staple cartridge 58 can be fixedly retained within the channel member 56 and the entire reload assembly 40 can be replaced to facilitate reuse of the stapling device 10. Although the cartridge assembly 52 is shown to pivot towards the anvil 50, it is envisioned that the cartridge assembly 52 could be stationary and the anvil 50 could pivot towards the cartridge assembly 52.

The mounting assembly 44 includes a first mounting member 60 and a second mounting member 62 that are secured together with posts 64 to define an enclosed channel 66 between the first and second mounting members 60 and 62, respectively. In aspects of the disclosure the posts 64 are formed on the first mounting member 60 and are received in openings 62a formed in the second mounting member 62. The second mounting member 62 defines bores 68 that receive the pivot members 54 to secure the mounting assembly 44 to a proximal end of the tool assembly 16. More specifically, the channel member 56 of the cartridge assembly 52 includes a proximal portion that defines bores 70 that receive the pivot members 54. The pivot members 54 extend through the bores 70 in the proximal portion of the channel member 56 and into the bores 68 of the second mounting member 62 to pivotably secure the cartridge assembly 52 to the mounting assembly 44. The pivot members 54 also extend through openings (not shown) in a proximal portion of the anvil assembly 50 to secure the anvil assembly 50 to the mounting assembly 44. The proximal portion of the anvil assembly 50 includes a proximally extending bracket 72 that defines an opening 72a, the function of which is described in further detail below.

The proximal body portion 42 of the reload assembly 40 includes a housing (80a, 80b), a drive assembly 82, an articulation mechanism 84 (FIG. 7), and a cylindrical casing 85. The housing is formed from half-sections 80a and 80b that are received within the casing 85 and are secured together to define internal channels that facilitate longitudinal movement of the drive assembly 82 and the articulation mechanism 84 within the housing 80. Each of the housing half-sections 80a and 80b includes a distal portion that defines a stepped cutout 86 (only one is shown). The first half-section 80a includes a proximal portion 88 that is configured to be releasably coupled to the distal portion of the elongate body 14 (FIG. 2). For a more detailed description of a reload assembly having a proximal body portion including a housing that is configured to releasably engage a body portion of an exemplary surgical stapling device, see U.S. Pat. No. 8,132,706 (hereinafter “the '706 Patent”).

Each of the mounting members 60 and 62 includes a pivot member 90 (only one is shown). The pivot members 90 are coaxial and define an articulation axis “Z” (FIG. 1) about which the tool assembly 16 articulates. The mounting assembly 44 also includes first and second pivot plates 92a and 92b. Each of the pivot plates 92a and 92b includes a body having a stepped configuration that corresponds to the configuration of the stepped cutouts 86 formed in the housing half-sections 80a and 80b. Each of the pivot plates 92a and 92b also includes a distal portion that defines a bore 94 that receives one of the pivot members 90 of the mounting members 60 and 62. The pivot plates 92a and 92b are received in the respective stepped cutouts 86 of the housing half-sections 80a and 80b and the pivot members 90 of the first and second mounting members 60 and 62 are received within the respective bores 94 of the pivot plates 92a and 92b to secure the mounting assembly 44 and tool assembly 16 to the proximal body portion 42 of the reload assembly 40 for pivotable movement about the articulation axis “Z”. The pivot member 90 on the mounting member 60 is also received in the opening 72a of the bracket 72 of the anvil assembly 50 to pivotally secure the tool assembly 16 to the proximal body portion 42 of the reload assembly 40.

The articulation mechanism 84 (FIG. 7) of the reload assembly 40 includes a proximal drive link 96, a distal drive link 98, a proximal driven link 100, and a distal driven link 102. The proximal drive link 96 is elongated and includes a proximal portion 104 that is configured to engage an articulation drive member (not shown) in the elongate body 14 of the stapling device 10 (FIG. 1). In aspects of the disclosure, the proximal portion 104 of the proximal drive link 96 includes a hook portion 106 that is configured to engage the articulation drive member (not shown) in the elongate body 14 of the stapling device 10. Alternately, other engagement configurations or devices are envisioned. The proximal drive link 96 also includes a distal guide portion 108 of increased width that includes a planar inner surface 108a that is positioned adjacent the drive assembly 82 to confine movement of the drive assembly as described below. In aspects of the disclosure, the distal guide portion 108 of the proximal drive link 96 is fixedly secured to the proximal portion of the proximal drive link 96 such as by welding. It is also envisioned that the proximal drive link 96 including the proximal portion 104 and the distal guide portion 108 can be integrally formed as a monolithic structure. In aspects of the disclosure, the proximal and distal drive links 96 and 98 including the distal guide portion 108 are formed of a rigid material that resists outward deformation of the drive assembly 82.

The distal drive link 98 is short as compared to the proximal drive link 96 and includes a distal portion that has an inner guide surface 98a that is positioned to guide movement of the drive assembly 82 as the drive assembly 82 bends about the articulation axis “Z” as described in further detail below. The inner guide surface may be curved. The distal drive link 98 has a proximal portion that is coupled to the distal guide portion 108 of the proximal drive link 96 by a pivot member 110. In aspects of the disclosure, the distal guide portion 108 defines a slot 112 that receives the proximal portion of the distal drive link 98 and the pivot member 110 extends through the slot 112 and into an opening 114 in the distal drive link 98 to pivotably couple the proximal drive link 96 to the distal drive link 98. The distal portion of the distal drive link 98 is coupled to one side of the first and second mounting members 60 and 62 at a position spaced outwardly of the pivot axis “Z”. In aspects of the disclosure, the distal portion of the distal drive link 98 defines an opening 99 that receives one of the posts 64 of the first mounting member 60 such that the distal portion of the distal drive link 98 is positioned between the first and second mounting members 60 and 62 and is pivotable about the post 64.

The proximal driven link 100 has a configuration like that of the distal guide portion 100 of the proximal drive link 96 and includes a planar inner surface 100a that is positioned adjacent the drive assembly 82 to confine movement of the drive assembly 82 as described below. The proximal driven link 100 has a distal portion that is coupled to the proximal portion of the distal driven link 102 by a pivot member 118. In aspects of the disclosure, the proximal driven link 100 defines a slot 116 that receives the proximal portion of the distal driven link 102 and the pivot member 118 extends through the slot 116 and into an opening 120 in the distal driven link 102 to pivotably couple the proximal driven link 100 to the distal driven link 102. The distal portion of the distal driven link 102 is coupled to the other side of the first and second mounting members 60 and 62 at a position spaced outwardly of the pivot axis “Z”. In aspects of the disclosure, the distal portion of the distal driven link 102 defines an opening 120 that receives the other of the posts 64 of the first mounting member 60 such that the distal portion of the distal driven link 102 is positioned between the first and second mounting members 60 and 62 and is pivotable about the post 64. The distal driven link 102 includes a distal portion that has inner guide surface 102a that is positioned to guide movement of the drive assembly 82 as the drive assembly 82 bends about the articulation axis “Z” as described in further detail below. The inner guide surface 102a may be curved. In aspects of the disclosure, the proximal and distal driven links 100, 102 are formed of a rigid material that resists outward deformation of the drive assembly 82.

The proximal drive link 96 and the proximal driven link 100 are received within channels defined between the first and second half-sections 80a and 80b of the housing and are enclosed by the casing 85 such that the links 96 and 100 are confined to linear movement between the first and second half-sections 80a and 80b of the housing proximal body portion 42. The proximal drive link 96 is driven linearly by the articulation drive member (not shown) of the elongate body 14 (FIG. 1) to advance (or retract) the distal drive link 98 and pivot the distal drive link 98 within the slot 112 in the distal guide portion 108. When the distal drive link 98 advances, the tool assembly 16 is pivoted about the articulation axis “Z”. When the tool assembly 16 pivots, the distal driven link 102 pivots and moves longitudinally within the slot 116 of the proximal driven link 100 and the proximal driven link 100 moves within the channel defined between the first and second half-sections 80a and 80b of the housing of the proximal body portion 42.

FIG. 4 illustrates the drive assembly 82 of the surgical device 10 which includes a flexible drive beam 140 and a clamp member 142. The flexible drive beam 140 has a proximal portion and a distal portion. The proximal portion of the drive beam 140 is coupled to a control rod (not shown) within the elongate body 14 (FIG. 1) of the stapling device 10 such that the drive assembly 82 is movable in response to movement of the control rod between retracted and advanced positions. In aspects of the disclosure, the flexible drive beam 140 is formed from stacked sheets or laminates and bends about the articulation axis “Z” (FIG. 2) when the tool assembly 16 is in an articulated position and the surgical device 10 is fired.

The clamp member 142 of the drive assembly 82 is secured to the distal portion of the drive beam 140 and is movable between retracted and advanced positions within the tool assembly 16 when the drive assembly 82 is moved between its retracted and advanced positions to actuate the tool assembly 16. In aspects of the disclosure, the clamp member 142 of the drive assembly 82 has an I-beam configuration and supports a knife blade 142a. In the retracted position, the clamp member is positioned in a proximal portion of the tool assembly 16 and the flexible drive beam 140 extends through the channel 66 in the mounting assembly 44. For a detailed description of the construction and operation of the drive assembly 82, see the '706 Patent.

The reload assembly 40 includes flexible stabilizing members 150, 152 positioned on each side of the flexible drive beam 140. Each of the flexible stabilizing members 150 and 152 extends from the proximal body portion 42 through the channel 66 defined by the mounting assembly 44. Each of the flexible stabilizing members 150, 152 has a distal end coupled to the mounting assembly 44 and a proximal end received within the housing 44 of the reload assembly 40 for sliding movement. In aspects of the disclosure, the distal ends of the of the stabilizing members 150 and 152 have outturned ends that are received within a cutout 154 (FIG. 7) formed in the mounting assembly 44.

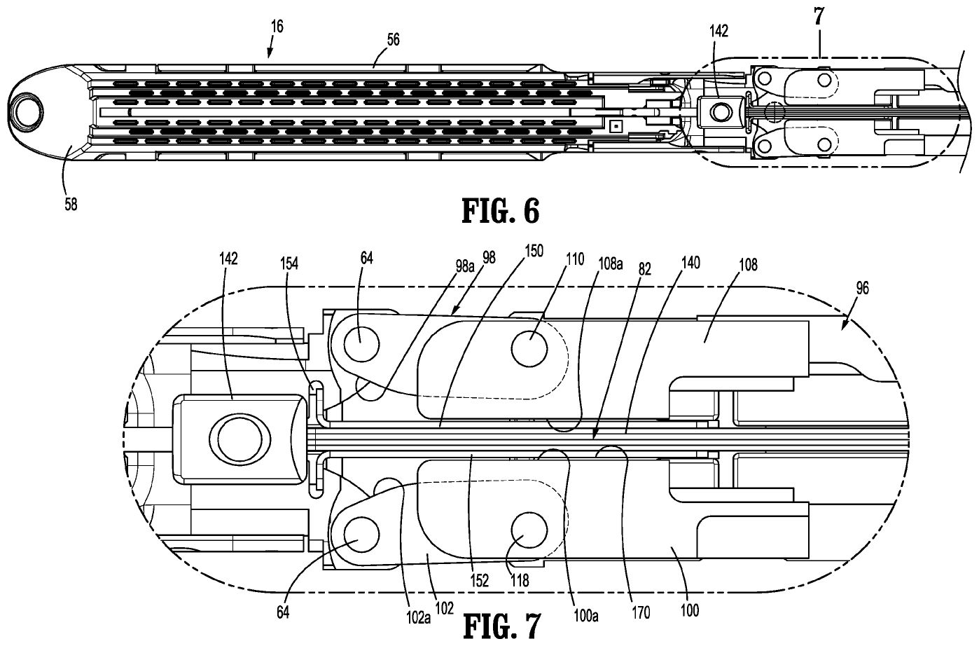

FIGS. 6 and 7 illustrate the reload assembly 40 with the tool assembly 16 in a non-articulated position. In the non-articulated position, the proximal drive link 96 is in an intermediate position and the distal drive link 98 and the distal driven link 102 are positioned in intermediate positions such that the tool assembly 16 is retained in the non-articulated position. In this position, the planar inner surfaces 108a and 100a of the proximal drive link 96 and the proximal driven link 100 are aligned with each other to define a guide channel 170 (FIG. 7) between the links 96 and 100. The flexible drive beam 140 of the drive assembly 82 extends through the channel 170 and through the channel 66 in the mounting assembly 44 towards the tool assembly 16.

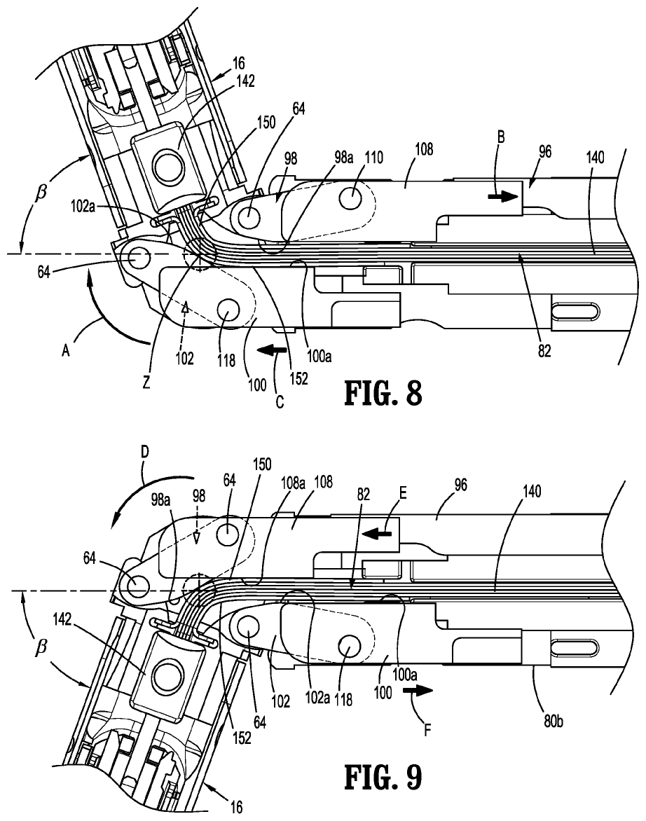

FIG. 8 illustrates the tool assembly 16 pivoted in a first direction indicated by arrow “A” to an angle “B”. When the proximal drive link 96 is retracted in the direction of arrow “B”, the distal drive link 98 is pulled proximally to pivot the tool assembly 16 in the direction of arrow “A”. As the tool assembly 16 pivots in the direction of arrow “A”, the distal driven link 102 is pulled distally and pivots inwardly towards the articulation axis “Z” to support the flexible drive beam 140 as it bends about the articulation axis “Z”. As illustrated, the proximal driven link 100 moves distally in the direction of arrow “C” such that the planar inner surface 100a moves distally to further support the flexible drive beam 140 at a position adjacent the pivot axis “Z”. This provides added support to the flexible drive beam 140 to minimize the likelihood of buckling and allow facilitate greater degrees of articulation.

FIG. 9 illustrates the tool assembly 16 pivoted in a second direction indicated by arrow “D” to an angle “B”. When the proximal drive link 96 is advanced in the direction of arrow “E”, the distal drive link 98 is pushed distally to pivot the tool assembly 16 in the direction of arrow “D” and the planar inner surface 108a of the proximal drive link 96 moves distally to support the flexible drive beam 140 adjacent the articulation axis “Z”. As the tool assembly 16 pivots in the direction of arrow “D”, the distal driven link 102 is pushed proximally and pivots inwardly towards the flexible drive beam 140. As illustrated, the proximal driven link 100 moves proximally in the direction of arrow “F” such that the planar inner surface 100a moves proximally to further support the flexible drive beam 140 at a position proximal of the planar inner surface 108a of the proximal drive link 96. This provides added support to the flexible drive beam 140 to minimize the likelihood of buckling and allow facilitate greater degrees of articulation.

When the drive assembly 82 is advanced to fire the surgical device 10 (FIG. 1) with the tool assembly 16 is in an articulated position, the proximal and distal drive links 96 and 98 and the proximal and distal driven links 100 and 102 move along an outer surface of the flexible drive beam 140 of the drive assembly 82 to stabilize the flexible drive beam 140 around the articulation axis “Z”. With the above-described articulation mechanism 84, the tool assembly 16 can articulate over an angle “β” of 70 degrees or more in each direction.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A surgical device comprising:
an elongate body defining a first longitudinal axis and having a proximal portion and a distal portion;
a tool assembly defining a second longitudinal axis, the tool assembly supported on the distal portion of the elongate body for pivotal movement about an articulation axis between a non-articulated position and articulated positions, the articulation axis being transverse to the first and second longitudinal axes;
a drive assembly including a flexible drive beam and a clamp member, the flexible drive beam having a proximal portion and a distal portion, the clamp member supported on the distal portion of the flexible drive beam and received within the tool assembly, the drive assembly movable between a retracted position and an advanced position to move the clamp member through the tool assembly; and
an articulation mechanism including a proximal drive link, a distal drive link, a proximal driven link, and a distal driven link, the proximal drive link having a planar inner surface, a proximal portion, and a distal portion, the distal drive link having a proximal portion pivotally coupled to the distal portion of the proximal drive link and a distal portion pivotally coupled to the tool assembly, the proximal driven link having a planar inner surface, a proximal portion, and a distal portion, the distal driven link having a proximal portion pivotally coupled to the proximal portion of the proximal driven link and a distal portion pivotally coupled to the tool assembly, wherein the proximal drive link is movable from an intermediate position to an advanced position to articulate the tool assembly about the articulation axis in a first direction and movable from the intermediate position to a retracted position to articulate the tool assembly about the articulation axis in a second direction, wherein the planar inner surfaces of the proximal drive link and the proximal driven link define a channel through which the flexible drive beam moves when the drive assembly is moved between the retracted and advanced positions.

2. The surgical device of claim 1, wherein the proximal drive link and the proximal driven link are confined to linear movement within the elongate body.

3. The surgical device of claim 2, wherein the planar inner surface of the proximal drive link is positioned to engage the flexible drive beam adjacent the articulation axis when the tool assembly is articulated in the first direction.

4. The surgical device of claim 3, wherein the planar inner surface of the proximal driven link is positioned to engage the flexible drive beam adjacent the articulation axis when the tool assembly is articulated in the second direction.

5. The surgical device of claim 4, wherein the distal drive link and the distal driven link have inner guide surfaces, the inner guide surface of the distal drive link positioned to engage the flexible drive beam adjacent the articulation axis when the tool assembly is articulated in the first direction.

6. The surgical device of claim 5, wherein the inner guide surface of the distal driven link is positioned to engage the flexible drive beam adjacent the articulation axis when the tool assembly is articulated in the second direction.

7. The surgical device of claim 1, wherein the distal drive link and the proximal drive link are formed from a rigid material.

8. The surgical device of claim 1, further including flexible stabilizing members positioned on each side of the flexible drive beam.

9. The surgical device of claim 8, wherein each of the flexible stabilizing members has a distal end coupled to the tool assembly and a proximal end received within the elongate body.

10. The surgical device of claim 1, further including a handle assembly coupled to the proximal portion of the elongate body.

11. The surgical device of claim 1, further including a mounting assembly fixedly coupled to the tool assembly and pivotally coupled to the elongate body.

12. The surgical device of claim 11, wherein the distal portions of the distal drive link and the distal driven link are pivotally coupled to the mounting assembly.

13. The surgical device of claim 12, wherein the mounting assembly defines a channel, and the flexible drive beam extends through the channel of the mounting assembly.

14. The surgical device of claim 1, wherein the proximal drive link and the proximal driven link define slots, and the distal drive link and the distal driven link are at least partly received within the slots.

15. A reload assembly comprising:
a proximal body portion defining a first longitudinal axis and having a proximal portion and a distal portion, the proximal body portion configured to releasably engage a surgical device;
a tool assembly defining a second longitudinal axis, the tool assembly supported on the distal portion of the proximal body portion for pivotal movement about an articulation axis between a non-articulated position and articulated positions, the articulation axis being transverse to the first and second longitudinal axes;
a drive assembly including a flexible drive beam and an I-beam, the flexible drive beam having a proximal portion and a distal portion, the I-beam supported on the distal portion of the flexible drive beam and received within the tool assembly, the drive assembly movable between a retracted position and an advanced position to move the I-beam through the tool assembly; and
an articulation mechanism including a proximal drive link, a distal drive link, a proximal driven link, and a distal driven link, the proximal drive link having a planar inner surface, a proximal portion, and a distal portion, the distal drive link having a proximal portion pivotally coupled to the distal portion of the proximal drive link and a distal portion pivotally coupled to the tool assembly, the proximal driven link having a planar inner surface, a proximal portion, and a distal portion, the distal driven link having a proximal portion pivotally coupled to the proximal portion of the proximal driven link and a distal portion pivotally coupled to the tool assembly, wherein the proximal drive link is movable from an intermediate position to an advanced position to articulate the tool assembly about the articulation axis in a first direction and movable from the intermediate position to a retracted position to articulate the tool assembly about the articulation axis in a second direction, wherein the planar inner surfaces of the proximal drive link and the proximal driven link define a channel through which the flexible drive beam moves when the drive assembly is moved between the retracted and advanced positions.

16. The reload assembly of claim 15, wherein the planar inner surface of the proximal drive link is positioned to engage the flexible drive beam adjacent the articulation axis when the tool assembly is articulated in the first direction.

17. The reload assembly of claim 16, wherein the planar inner surface of the proximal driven link is positioned to engage the flexible drive beam adjacent the articulation axis when the tool assembly is articulated in the second direction.

18. The reload assembly of claim 17, wherein the distal drive link and the distal driven link have inner guide surfaces, the inner guide surface of the distal drive link positioned to engage the flexible drive beam adjacent the articulation axis when the tool assembly is articulated in the first direction.

19. The reload assembly of claim 18, wherein the inner guide surface of the distal driven link is positioned to engage the flexible drive beam adjacent the articulation axis when the tool assembly is articulated in the second direction.

20. A surgical device comprising:
an elongate body defining a first longitudinal axis and having a proximal portion and a distal portion;
a tool assembly defining a second longitudinal axis, the tool assembly supported on the distal portion of the elongate body for pivotal movement about an articulation axis between a non-articulated position and articulated position, the articulation axis being transverse to the first and second longitudinal axes;
a drive assembly including a flexible drive beam and a clamp member, the flexible drive beam having a proximal portion and a distal portion, the clamp member supported on the distal portion of the flexible drive beam and received within the tool assembly, the drive assembly movable between a retracted position and an advanced position to move the clamp member through the tool assembly; and
an articulation mechanism including a drive link and a driven link, the drive link having a distal portion, a proximal portion, and a planar inner surface extending between the proximal and distal portions, the distal portion pivotally coupled to the tool assembly, the driven link having a proximal portion, a distal portion, and a planar inner surface extending between the proximal and distal portion of the driven link, the planar inner surfaces of the drive link and the driven link defining a linear channel through which the flexible drive beam moves when the drive assembly is moved between the retracted and advanced positions, wherein the drive link is movable from an intermediate position to an advanced position to articulate the tool assembly about the articulation axis in a first direction and movable from the intermediate position to a retracted position to articulate the tool assembly about the articulation axis in a second direction.
